(19) United States
(12) Patent Application Publication
(10) Pub. No.: US 2021/0022581 A1
(43) Pub. Date: Jan. 28, 2021

(54) Multi-Use Sanitizing Mop and Sprayer

(71) Applicant: Thane IP Limited, London (GB)

(72) Inventor: Jelenko Piksa, Toronto (CA)

(73) Assignee: Thane IP Limited, London (GB)

(21) Appl. No.: 16/672,871

(22) Filed: Nov. 4, 2019

(30) Foreign Application Priority Data
Jul. 22, 2019 (IB) PCTIB2019000861

(51) Int. Cl. A47L 13/22; C25B 1/04; C02F 1/461

(52) U.S. Cl. CPC A47L 13/22; C25B 1/04; C02F 1/46109; C02F 2201/4618; C02F 2001/46157; C02F 2001/46171; C02F 2201/005

(57) ABSTRACT

A versatile sanitizing and cleaning apparatus (10) has a reservoir (30), a removable base (40) at the bottom of the reservoir having electrodes (52, 54) that extend up into the reservoir (30) for electrolyzing water within the reservoir, and an opening (32) at the top end of the reservoir. A cap (60) having a valve (61) that is biased to the normally-closed position screws to the top of the reservoir (30), allowing the reservoir to be turned upside-down and inserted into a receiving portion (22) of a mop, thus allowing the mop (12) to use electrolyzed water for cleaning. Alternatively, instead of being used with the cap (60) and mop (12), the reservoir (30) and base (40) can be mated with a hand sprayer (64) allowing the reservoir (30), base (40), and hand sprayer (64) to be used as a spray bottle (69) to spray electrolyzed water for cleaning purposes.

### CROSS-REFERENCE TO RELATED APPLICATIONS

This application is a continuation of PCT International Application No. PCT/IB2019/000861 filed Jul. 22, 2019.

### BACKGROUND OF THE INVENTION

### 1. Field of the Invention

This invention relates to the field of sanitizing and cleaning apparati. More particularly, this invention relates to the field of a versatile multi-use sanitizing mop and sprayer, in which a container that includes electrodes for making sanitizing electrolyzed water can be used as both a sanitizing water reservoir in a mop and as a sanitizing water reservoir for a sprayer bottle.

### 2. Description of Related Art

The sanitizing properties of electrolyzed water, or more specifically an electrolyzed salt solution, are known. As used herein the term “electrolyzed water” is a general term referring to any solution produced by the electrolysis of water containing an electrolyte such as sodium chloride, potassium chloride or dilute hydrochloric acid. When the salt solution is sodium chloride which is commonly referred to as table salt dissolved in water, the saline solution is electrolyzed to produce hypochlorous acid, sodium hypochlorite, and sodium hydroxide. Various prior art systems employ semi-permeable membranes to mechanically isolate the anode and cathode of an electrolyzing cell while permitting ion transfer between the anode and the cathode to complete the electrical circuit. At the anode, chloride ions oxidize to form chlorine, which then combines with water to make hypochlorous acid. The hypochlorous acid is drawn off the anode cell. At the cathode, water is reduced into hydrogen gas and hydroxide ions. The hydroxide ions combine with sodium ions to make sodium hydroxide. The sodium hydroxide is then drawn off the cathode cell. Industrial systems can make these products continuously.

Other systems exist for creating small batches of electrolyzed water such as for home cleaning. U.S. Patent publication No. 2016/0330968 by Owens et al. discloses a system for creating small batches of cleaning and/or sanitizing solutions in the same portable dispensing container in which the products are created, thus eliminating the need to produce the desired products in one volumetric system and subsequently transfer desired products into another vessel in which the products can then be applied to surfaces to be cleaned. The portable dispensing container can be a spray bottle with the electrodes built into the spray bottle. Owens et al. disclose various salt and vinegar solutions that can be used, together with voltages and waveforms, in creating the electrolyzed water.

U.S. Pat. No. 6,926,819 to Nakamura et al. and U.S. Patent Publication No. 2004/0011665 by Koizumi et al. also disclose small batch systems for creating electrolyzed water, with the electrolyzing vessel being a spray bottle. Those references provide additional teachings regarding structures and processes for creating electrolyzed water for cleaning and sanitizing purposes. All of those references are incorporated herein for their teachings of structures and processes for creating electrolyzed water for cleaning and/or sanitizing purposes.

### SUMMARY OF THE INVENTION

The present invention is of a versatile sanitizing and cleaning apparatus in which electrolyzed water is created in an electrolyzing vessel, and the electrolyzing vessel can then be conveniently used as either the reservoir of a spray pump or can be placed into a receiving portion of a mop with the mop then releasing the electrolyzed water from the electrolyzing vessel and spraying the electrolyzed water onto a floor surface to be cleaned.

In one embodiment and aspect the invention includes a reservoir having a first (top) threaded opening at a first (top) portion of the reservoir and a second (bottom) threaded opening at a second (bottom) portion of the reservoir. A base is removably screwed to the bottom opening and forms a watertight seal therewith. The base includes two electrodes, an anode and a cathode, that extend up into the reservoir. In one embodiment the electrodes are each formed into respective mesh material that is then formed into respective tubular structures having circular cross-sections of different diameters, with one electrode disposed inside the other electrode as concentric tubes, with the inner electrode having a smaller circular cross-section that the outer electrode. The base has an electrical input port for receiving a DC voltage from an AC/DC power converter, a timer, and a pushbutton for activating the timer. The timer allows electrical energy to flow from the electrical input port to the electrodes for electrolyzing a salt solution within the reservoir for a predefined amount of time, such as 10 minutes. Although preferably the base contains only timer electronics to keep the electrolyzing vessel simple and lightweight, the base can optionally include the AC/DC power supply, a DC/DC converter for converting the input voltage to a different voltage, a waveform control for applying different waveforms to the electrodes, or other electrical controls for powering and controlling the creation of electrolyzed water at the electrodes. The reservoir and the base, optionally with a cap or sprayer described below, function as an electrolyzing vessel.

The top opening can be screwed to a hand sprayer assembly so that the reservoir, base, and hand sprayer assembly together form a portable spray bottle that the user can use to spray electrolyzed water for cleaning and sanitizing purposes.

Alternatively, the top opening can be screwed to a cap that has a valve that is biased to the normally-closed position, such that the reservoir, base, and cap can be turned cap-side down without water substantially spilling from the reservoir. This electrolyzing vessel can then be inserted into and mated with a reservoir receiving portion of a mop without water spilling from the electrolyzing vessel. The mop has a rotatable collar that includes a mating tab such that as the collar is rotated the mating tab slides into a circular groove in the bottom of the base, thus affirmatively holding the electrolyzing vessel to the reservoir receiving portion of the mop. The reservoir receiving portion has a protuberance or other valve actuator that opens the biased valve in the cap, thus allowing water to flow into the reservoir receiving portion, from which it can be sprayed out in front of the mop by a squeeze handle on the mop which operates a spray pump located in the reservoir receiving portion.

Preferably the base and reservoir have cross-sectional profiles that are identical where the base and the reservoir meet, such that the base and the reservoir peripheral walls flow smoothly together aesthetically without substantial discontinuities therebetween.

The base and the reservoir preferably have respective vertically extending concave grooves formed therein for partially receiving the mop handle, such that the mop handle is partially nestled into the reservoir and base structures.

The reservoir and the cap also preferably have appropriate one-way valves such that: (a) the gasses created as part of the electrolyzing process, principally hydrogen gas, can be vented to the atmosphere and will not cause the electrolyzing vessel to explode; (b) a vacuum is not created within the reservoir as electrolyzed water is drawn from it which would prevent water from flowing out of the reservoir to the mop's floor spray pump.

In another embodiment the electrolyzing vessel is integrated into the mop and not necessarily easily removable. In this embodiment the mop includes a liquid reservoir with electrodes integrated into the reservoir for electrolyzing water or other electrolyte solution in the reservoir. The user externally mixes a salt solution, a salt/vinegar solution, or other solution, then pours the prepared solution into the vessel and turns on the activation switch. Alternatively the user could add water and salt/vinegar directly to the reservoir. The electrolyzed solution is then created within the mop's reservoir. As with the first embodiment, a timer can be integrated into the vessel or other location within the mop for turning the electrolyzing current off after a predetermined amount of time when the electrolyzation is complete.

Exemplary embodiments of the invention will be further described below with reference to the drawings, in which like numbers refer to like parts. The drawing figures might not be to scale, and certain components may be shown in generalized or schematic form and identified by commercial designations in the interest of clarity and conciseness.

### BRIEF DESCRIPTION OF THE DRAWINGS

FIG. 1 is a front left perspective view of the sanitizing and cleaning apparatus according to one embodiment of the present invention;

FIG. 2 is a closeup view of the apparatus of FIG. 1 showing the electrolyzing vessel removed from the mop, together with the spray pump;

FIG. 3 is an exploded view of the electrolyzing vessel, and the spray pump assembly, of the apparatus of FIG. 1;

FIG. 4A is a top perspective view of the cap of the electrolyzing vessel of FIG. 3;

FIG. 4B is a bottom perspective view of the cap of the electrolyzing vessel of FIG. 3;

FIG. 5 is a top perspective view of the base of the electrolyzing vessel of FIG. 3;

FIG. 6 is a top perspective view of the electrolyzing vessel of FIG. 3 with the cap removed and the spray handle of FIG. 3 installed thereon, together with a power supply, being used to electrolyze water;

FIG. 7 is a top perspective view of the electrolyzing vessel of FIG. 3 together with a power supply, being used to electrolyze water;

FIG. 8 is a top perspective view of the mop of FIG. 1 with the electrolyzing vessel not yet secured to the mop handle; and

FIG. 9 is a top perspective view of the mop of FIG. 1 with the electrolyzing vessel now secured to the mop handle.

### DETAILED DESCRIPTION OF THE PREFERRED EMBODIMENTS

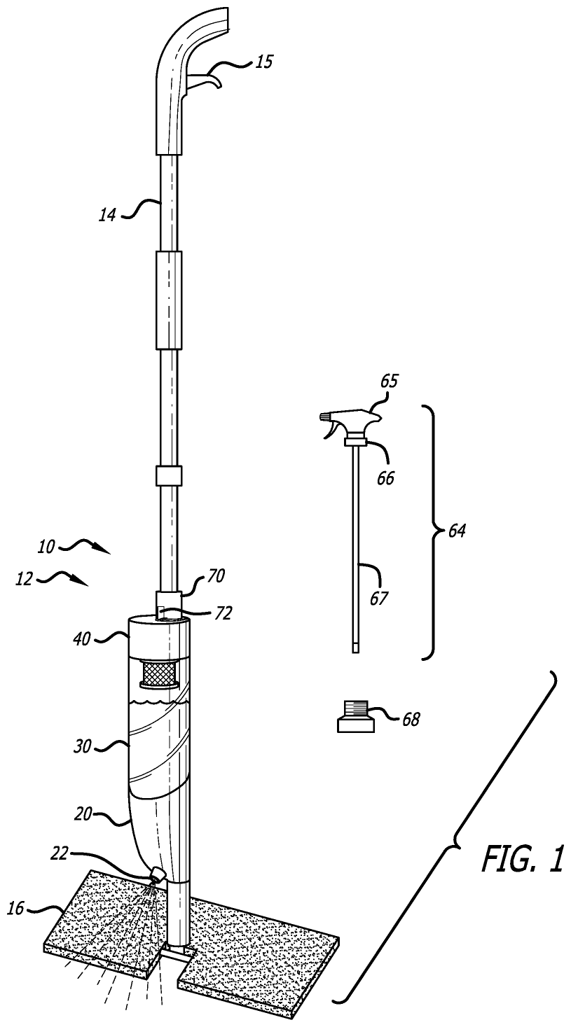

FIG. 1 is a front left perspective view of the sanitizing and cleaning apparatus according to one embodiment of the present invention. The apparatus or system 10 includes a mop 12 having an elongate mop handle 14, a reservoir receiving portion 20 mounted to the mop handle 14 above mop head 16 near the lower end of handle 14 when the mop 10 is held in an upright position, a trigger 15 at the mop handle for powering a spray pump (not shown) contained within reservoir receiving portion 20, and an adjustable spray nozzle 22 through which electrolyzed water is dispensed onto a floor surface after being withdrawn from an interior of reservoir 30 and pumped by the user squeezing trigger 15. Reservoir receiving portion 20 receives and holds the electrolyzing vessel which includes reservoir 30 and base 40.

A spray pump assembly 64 or simply spray pump includes a hand-operated spray handle 65, a threaded collar 66, and a tube 67. The spray pump assembly 64 can also include adapter 68 that allows standard spray pump assembly 64 to be threadingly mated to the electrolyzing vessel which includes reservoir 30 and base 40.

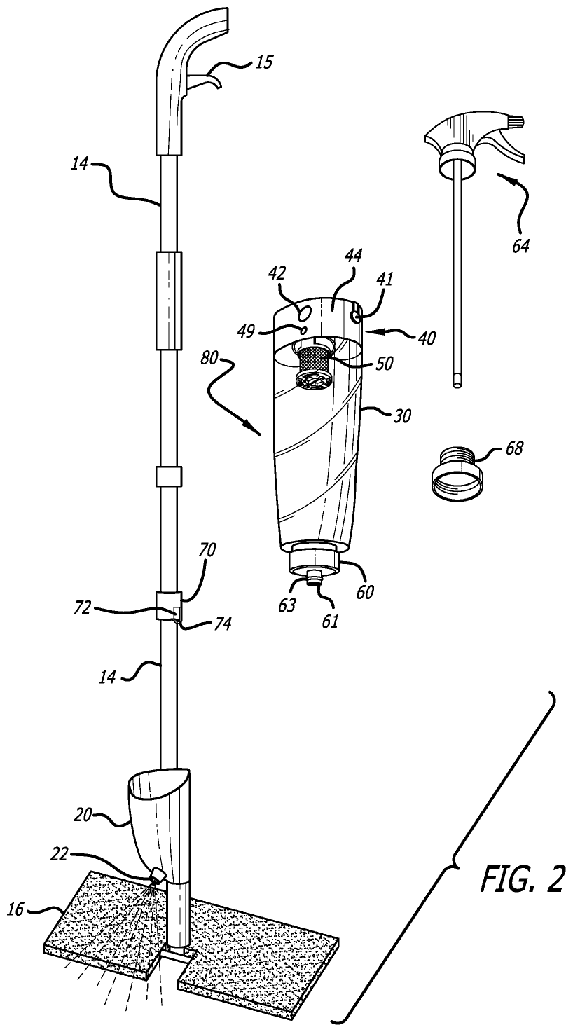

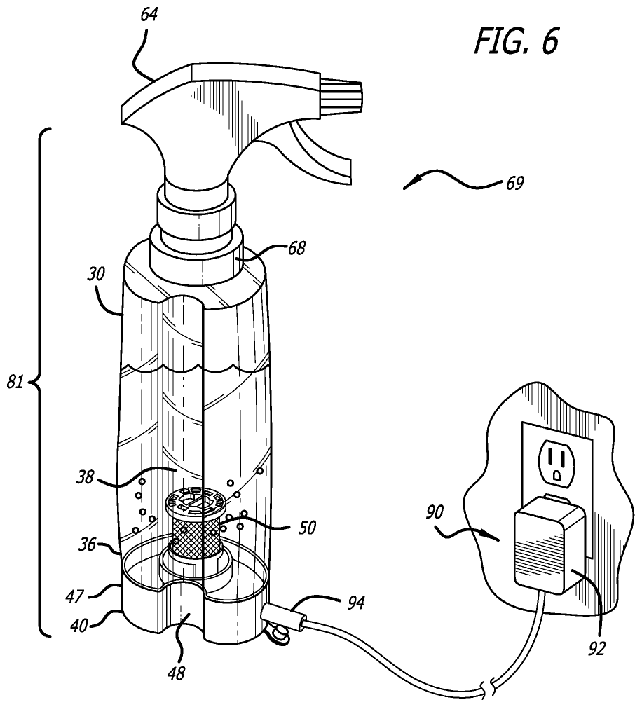

FIG. 2 is a closeup view of the apparatus of FIG. 1 showing the electrolyzing vessel removed from the mop 12, together with the spray pump 64. The electrolyzing vessel includes reservoir 30, base 40, and cap 60. Base 40 includes a base housing 44, an electrical input port 41 for receiving electrical input power such as an input voltage, an activation button 42, an indicator light 49, and electrode assembly 50 which extends from base 40 up through a threaded second (bottom) opening in reservoir 30. Electrode assembly 50 is visible in the figure through the preferably transparent wall of reservoir 30. Base 40 includes a timer (not shown) that is activated by activation switch 42 to connect power from electrical input port 41 to the two electrodes in electrode assembly 50. Activation switch 42 is an activation button in this embodiment. When power is connected to electrode assembly 50, the indicator light 49 such as a red LED indicates that the unit is on. The timer turns the power off after a predetermined amount of time such as 10 minutes and thus acts as a simple controller for controlling the electrolyzation process. In order to electrolyze water in the electrolyzation vessel, the user puts water into reservoir 30, preferably substantially filling the interior of reservoir 30, adds the correct amount of table salt or possibly other electrolyte such as potassium chloride, optionally shakes or stirs the vessel to ensure that the salt is completely dissolved, plugs the power supply into a wall socket as seen in FIG. 6, and presses activation button 42. In this embodiment the reservoir 30 should be filled with approximately 0.45 liter of water and approximately 3 grams of table salt should be added; the power supply is a 120 VAC/5 VDC power supply providing 5.0 VDC to base 40 which is connected directly to the electrodes 52/54 through the timer, and the predetermined amount of time is approximately 10 minutes. The unit automatically turns off after 10 minutes and the indicator LED 49 extinguishes thereby signaling to the user that the electrolyzing process is completed and the electrolyzed water is ready to use. In this context “complete” means that a desired amount of electrolyzation has occurred. Alternatively, instead of an indicator light such as a red LED that extinguishes when the electrolyzation is complete, the indicator light could be a light such as a green LED that illuminates when the electrolyzation is complete.

Optionally a small amount of a weak acid can be added for additional sanitizing and cleaning properties. Examples of suitable weak acids include vinegar which is a 5-20% acetic acid solution, citric acid, malic acid, or lactic acid. If vinegar having 5% acetic acid is used, then approximately 2.5 ml of vinegar can be mixed with 0.45 liters of water.

Different voltages and waveforms could be applied at the electrodes, with the amount of electrolyzation time adjusted accordingly. The base could include the AC/DC power converter, or a DC/DC power converter, and the controller in base 40 could include more complex control such as controlling the power applied to the electrodes in a more complex manner. For example, the voltage supplied to the electrodes could be a sawtooth wave, a square wave, increasing or decreasing in amplitude over time, and/or be current limited. The base could include more complex sensing and feedback mechanisms such as a pH sensor and feedback loop for controlling the pH of the resulting water, or sensing and controlling for concentrations of particular chemical constituents, or sensing and controlling for temperature or other parameters. The currently anticipated commercial embodiment, however, will merely apply 5.0 VDC to the electrodes for 10 minutes, with only a simple timer as the only controller within base 40. This will keep the base 40 lightweight and thus easy to handle, including when the electrolyzing vessel is used as a spray bottle as will be described below.

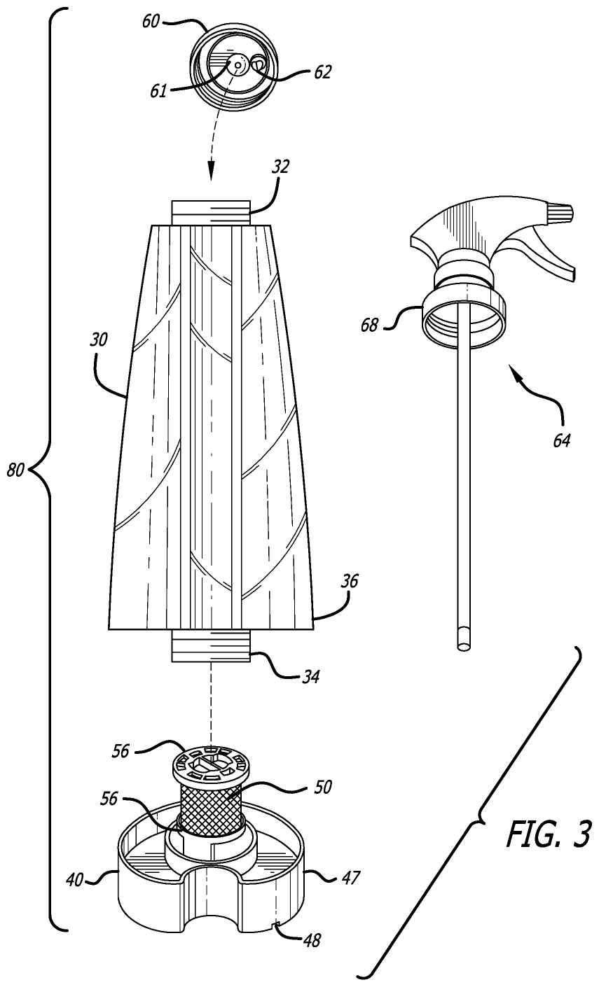

FIG. 3 is an exploded view of the first electrolyzing vessel 80, and the spray pump assembly 64, of the apparatus of FIG. 1. Electrolyzing vessel 80 defines a reservoir assembly that includes reservoir 30, base 40, and cap 60. Reservoir 30 has a first or top threaded opening 32 that threadingly mates with cap 60, and a second or bottom threaded opening 34 that threadingly mates with base 40. Reservoir 30 also has at least one peripheral wall 36 adjacent where the reservoir 30 mates with base 40, and base 40 has at least one peripheral wall 47 adjacent where base 40 mates with reservoir 30. Peripheral walls 36 and 47 have substantially identical shapes such that the reservoir peripheral wall 36 flows or transitions smoothly to base peripheral wall 47 substantially without any discontinuities therebetween. In this embodiment the cross-sections of peripheral walls 36 and 47 are non-circular and are suitable for the user to hold electrolyzing vessel 80 comfortably in his hand.

Cap 60 includes a biased valve 61 that is biased to a normally-closed position by a spring (not shown). As used herein the word “spring” is an umbrella term that encompasses any mechanical mechanism such as a steel coil spring or other resilient member that provides a bias force. Biased valve 61 allows reservoir 30 to be turned second opening down without liquid substantially spilling from the reservoir. Cap 60 also includes one-way air valve 62 that acts as a vacuum prevention valve. Vacuum prevention valve 62 allows air to enter reservoir 30 as electrolyzed water is drawn from it, thus preventing significant vacuum from building up inside reservoir 30 would prevent further electrolyzed water from being drawn from reservoir 30.

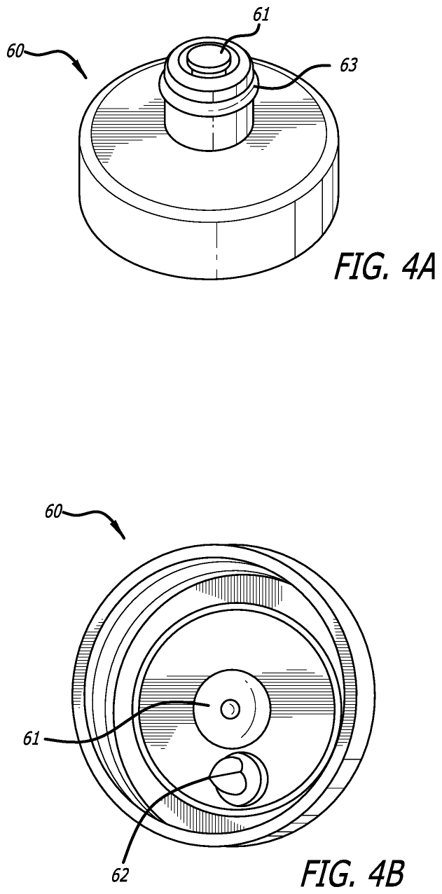

FIG. 4A is a top perspective view of the cap of the electrolyzing vessel of FIG. 3, showing biased valve 61 and O-ring 63 that helps cap 60 to form watertight seal with reservoir receiving section 20 of mop 12 without water spilling when valve 61 gets opened and water flows out of reservoir 30. Reservoir receiving portion 20 has a fixed pin, protuberance or other valve actuator (not shown) that overcomes the bias within biased valve 61 thus opening that valve when the reservoir 30 and its cap 60 are mated with reservoir receiving portion 20. This allows the electrolyzed water within reservoir 30 to flow into the reservoir receiving portion 20 from which it can be sprayed out in front of the mop by trigger 15 which operates a spray pump located in the reservoir receiving portion. In the anticipated commercial embodiment the valve actuator is a simple fixed pin that always opens biased valve 61 whenever electrolyzing vessel 80 is set into place within reservoir receiving portion 20. As the reservoir 30 is being seated within reservoir receiving portion 30, the O-ring first slides into a receiving tube (not shown) and forms a watertight seal with the tube before the pin engages and opens valve 61, thus preventing the electrolyzed water from spilling.

FIG. 4B is a bottom perspective view of cap 60 of the electrolyzing vessel 80 of FIG. 3, revealing the bottom of biased valve 61 and overpressure valve 62.

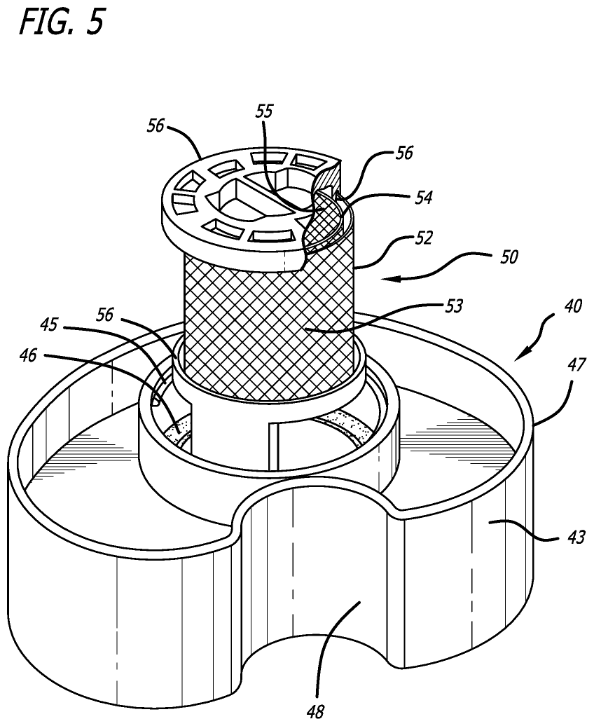

FIG. 5 is a top perspective view of base 40 of the electrolyzing vessel of FIG. 3 unscrewed from reservoir 30. An opening 45 having a female thread screws to and thereby mates with opening 34 having a corresponding male thread on reservoir 30. A silicone gasket 46 ensures a watertight seal between reservoir 30 and base 40. Base 40 can be repeatedly separated from reservoir 30 and re-mated thereto. Although it is preferably that base 40 be separable from reservoir 30 as described for cleaning and replacement part purposes, alternatively base 40 could be integral with reservoir 30. Electrode assembly 50 includes a first and outer electrode cross-section, and a second and inner electrode 54 also in the form of a second mesh formed into a second tubular structure having a second circular cross-section, and located inside the first electrode 52. The electrodes 52/54 thus form concentric tubes. Electrodes 52/54 are electrically and physically separated by an insulator 56.

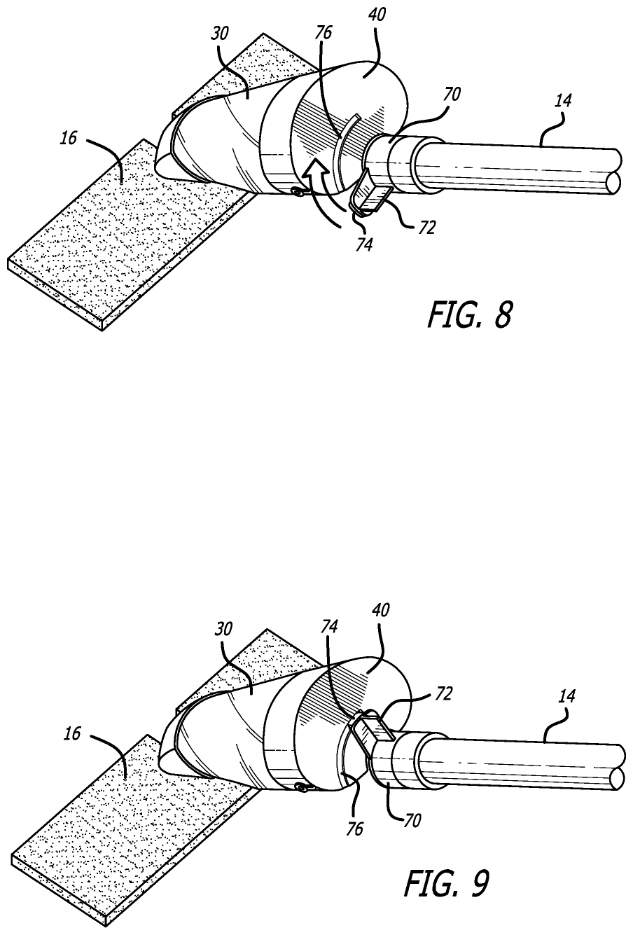

Base 40 has a concave groove 48 therein, and reservoir 30 has a concave groove 38 therein. These two concave grooves 48/38 are aligned. Their purpose is to partially receive mop handle 12 as seen best in FIGS. 8 and 9, thus helping to secure electrolyzing vessel 80 to mop handle 12.

Wires or other electrical conductors carry the electrolyzing voltage from electrical input port 41 to electrodes 52/54, in this embodiment through a switch controlled by the timer within base 40. Alternatively, the electrical power could be routed through a transformer, a wave shaper, a current limiter, or other electronics for controlling the voltage and other power parameters across electrodes 52/54. Still further, electrical input port 41 could receive input electrical power through other means including indirect means such as inductive power transfer. Regardless of what electronics may intervene between power input port 41 and electrodes 52/54, electrical power at power input port 41 powers the electrolyzing voltage and current across electrodes 52/54. Cathodes are generally defined as negatively charged electrodes, and anodes are generally defined as positively charged electrodes. Accordingly, electrode 52 is the anode and electrode 54 is the cathode if electrode 54 is at a lower electrical potential than electrode 52. In this embodiment it is not considered important which electrode 52/54 is used as the anode and which is used as the cathode.

FIG. 6 is a top perspective view of the electrolyzing vessel 80 of FIG. 3 with the cap 60 removed and the spray pump assembly 64 and adapter 68 of FIG. 3 installed thereon, together with power supply 90, being used to electrolyze water, defining a second and slightly different electrolyzing vessel 81. In this configuration electrolyzing vessel 30/40 together with spray pump assembly 64 and adapter 68 define a complete spray bottle 69 that can be filled with water and the desired salt and optionally vinegar, then plugged in to the wall socket and activated by pressing the activation button to begin the electrolyzing process. When the electrolyzation is complete as indicated by indicator light 49 turning off, the user unplugs power plug 94 from electrical input port 41 and begins using the spray bottle 81 to spray sanitizing electrolyzed water onto whatever surface the user desires to clean and sanitize.

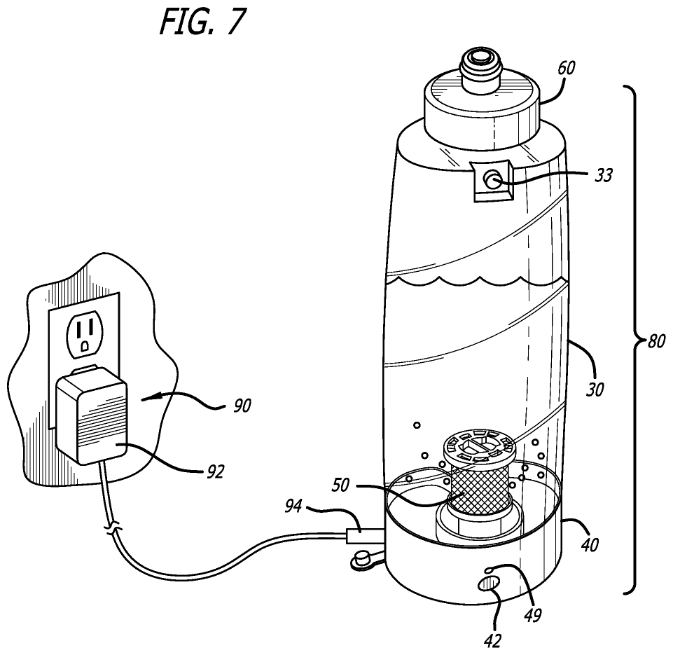

FIG. 7 is a top perspective view of the electrolyzing vessel 80 of FIG. 3 together with power supply 90, being used to electrolyze water. As with the configuration shown in FIG. 6 the user fills reservoir 30 with water, adds salt and optionally shakes or stirs electrolyzing vessel 80 to help fully dissolve the salt, then plugs the assembly in to the wall socket and activates the electrolyzation process by pressing the activation button. Reservoir 30 includes an pressure relief valve or overpressure valve 33 that allows gas to escape from the electrolyzing vessel 80 during electrolyzation so that the vessel does not explode or otherwise become overpressured. Once the electrolyzation process is complete as indicated by indicator light 49 turning off, the user unplugs power plug 94 from electrical input port 41 then turns electrolyzing vessel 80 upside down and places the unit cap end first into reservoir receiving portion 20 of mop 12, and clamps it into place as shown in FIGS. 8-9.

Power supply 90 can be a standard 120 VAC/5.0 VDC power supply, with power plug 94 being a standard 5.0 VDC power plug. Such power supplies are standard items that are widely available and inexpensive. Power supply 90 is thus easily and inexpensively replaced should that part fail.

FIG. 8 is a top perspective view of the mop 12 of FIG. 1 with the electrolyzing vessel 80 having been placed into reservoir receiving portion 20 but not yet secured to the mop handle 14. Collar 70 on mop handle 14 can rotate around mop handle 14. Collar 70 includes a push tab 72 that a user can press against with his thumb or finger, and an engagement tab 74 (FIG. 9) that engages circular slot 76 in base 40.

FIG. 9 is a top perspective view of the mop 12 of FIG. 1 with the electrolyzing vessel 80 now secured to the mop handle 12 by collar 70 including push tab 72 and engagement tab 74 being rotated to its clockwise-most position. In this position engagement tab 74 extends into groove 76 with a friction fit and holds base 40 thereto, thus securing electrolyzing vessel 80 to mop handle 12. Alternative means of securing electrolyzing vessel 80 to mop handle 12 could be used.

A versatile multi-use cleaning and sanitizing apparatus including an electrolyzing vessel having different configurations has thus been disclosed that allows a user to easily and conveniently make electrolyzed water and use that electrolyzed water either in a mop or a spray bottle for household cleaning and sanitizing purposes. The unit is lightweight and inexpensive, and is easily disassembled for cleaning or for parts replacement. The unit can use both a standardized hand spray pump assembly and a standardized power converter so that those parts can be easily and inexpensively replaced.

In another embodiment the electrolyzing vessel is integrated into the mop and not necessarily easily removable. In this embodiment the mop includes a liquid reservoir with electrodes integrated into the reservoir for electrolyzing water or other electrolyte solution in the reservoir, defining an electrolyzing vessel. The electrolyzing vessel is integrated into the mop or is otherwise held by a electrolyzing vessel holding portion of the mop. The user externally mixes a salt solution, a salt/vinegar solution, or other desired electrolyte solution, then pours the prepared solution into the vessel and turns on the activation switch. The vessel can include a liquid intake port for receiving the electrolyte solution, such as an opening with a watertight cap on it. The user then turns on an electrical switch or plugs the power supply in, thus beginning the electrolyzing process that takes place within the mop's reservoir. A timer can be integrated into the vessel or other location within the mop for turning the electrolyzing current off after a predetermined amount of time when the electrolyzation is complete, such as 10 minutes. As with the first embodiment, the electrolyzed water then flows out of an opening in the reservoir, and preferably to a hand-operated spray pump as in the prior embodiment. As with the prior embodiment the reservoir preferably has a one-way valve to prevent vacuum from building up. As with the first embodiment the mop includes a sprayer that receives the electrolyzed solution after the electrolyzed solution has been withdrawn from the first opening and sprays the electrolyzed solution away from the mop and onto the floor to be cleaned.

It will be understood that the terms “generally,” “approximately,” “about,” “substantially,” “aligned” and “smoothly” as used within the specification and the claims herein allow for a certain amount of variation from any exact dimensions, measurements, and arrangements, and that those terms should be understood within the context of the description and operation of the invention as disclosed herein.

It will further be understood that terms such as “top,” “bottom,” “above,” and “below” as used within the specification and the claims herein are terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise. Thus, unless expressly stated otherwise, each feature disclosed is one example only of a generic series of equivalent or similar features.

It will be appreciated that the term “present invention” as used herein should not be construed to mean that only a single invention having a single essential element or group of elements is presented. Similarly, it will also be appreciated that the term “present invention” encompasses a number of separate innovations which can each be considered separate inventions. Although the present invention has thus been described in detail with regard to the preferred embodiments and drawings thereof, it should be apparent to those skilled in the art that various adaptations and modifications of the present invention may be accomplished without departing from the spirit and the scope of the invention. Accordingly, it is to be understood that the detailed description and the accompanying drawings as set forth hereinabove are not intended to limit the breadth of the present invention, which should be inferred only from the following claims and their appropriately construed legal equivalents.

We claim:

1. A versatile sanitizing and cleaning apparatus comprising:
a reservoir having a first opening at a first portion thereof and a second opening at a second portion thereof;
a base that mates with the first opening of the reservoir to form a watertight seal with the reservoir, the base comprising;
a base housing;
an electrical input port for receiving input electrical power; and
first and second electrodes defining a cathode and an anode extending from the base into the reservoir when the reservoir first opening is mated to the base; and
wherein input electrical power applied at the electrical input port powers an electrical potential between the first and second electrodes for electrolyzing water within the reservoir;
a spray pump adapted for mating with the second opening of the reservoir such that the reservoir, the base, and the spray pump can be used together as a pump to spray liquid from the reservoir onto a surface to be cleaned;
a cap adapted for mating with the second opening of the reservoir, the cap having a biased valve such that when the cap is mated to the second opening of the reservoir the reservoir can be turned second opening down without liquid substantially spilling from the reservoir;
a mop comprising:
an elongate handle;
a mop head at a lower end of the elongate handle;
a reservoir receiving portion affixed to the elongate handle near the lower end thereof and above the mop head when the mop is held upright, the reservoir receiving portion adapted to receive and hold the reservoir; and
a valve actuator such that when the reservoir is received into the reservoir receiving section the valve actuator opens the biased valve within the cap such that liquid can flow from the reservoir to a floor surface being mopped by the mop head.

2. The apparatus of claim 1 wherein:
said reservoir comprises at least one peripheral wall adjacent a location where the reservoir mates with the base; and
said base has at least one peripheral wall adjacent a location where the base mates with the reservoir; and
the reservoir peripheral wall and the base peripheral wall have substantially identical shapes such that said reservoir peripheral wall transitions smoothly to said base peripheral wall when the reservoir and base are mated together.

3. The apparatus of claim 1 wherein both the reservoir and the base have respective concave grooves formed therein, the concave grooves receiving a portion of the elongate handle of the mop.

4. The apparatus of claim 1 wherein said base removably mates with said reservoir such that the base can be separated from the reservoir and thereafter mated with the base again.

5. The apparatus of claim 1 wherein the reservoir further comprises a pressure relief valve.

6. The apparatus of claim 1 wherein the base further comprises a timer and an associated activation switch for allowing electrical power to flow from the electrical input port to the electrodes for a predetermined amount of time.

7. A versatile sanitizing and cleaning apparatus comprising: a removable reservoir assembly comprising:
a reservoir having an interior for containing a liquid;
a pair of electrodes in contact with the liquid when the reservoir is substantially filled with the liquid;
an electrical input port in electrical communication with the electrodes; and
a valve in fluid communication with the reservoir interior, the valve having a spring that biases the valve toward a closed position; and
a mop comprising:
an elongate handle;
a mop head at a lower end of the elongate handle for mopping a floor surface;
a reservoir receiving portion affixed to the elongate handle near the lower end thereof and above the mop head when the mop is held upright, the reservoir receiving portion adapted to receive and hold the reservoir assembly, the reservoir assembly being removable from the reservoir receiving section;
a valve actuator for overcoming the bias and opening the valve; and
a spray pump such that when the valve is open and liquid flows from the reservoir liquid can be sprayed away from the mop and onto the floor surface.

8. The apparatus of claim 7 wherein said reservoir assembly further comprises:
a base that threadingly engages a first opening of the reservoir to form a watertight seal between the reservoir and the base;
wherein the electrodes are affixed to the base and extend up through the first reservoir opening into the reservoir when the base is threadingly engaged with the reservoir.

9. The apparatus of claim 7 wherein the electrodes comprise:
a first electrode in the form of a first mesh formed into a first tubular structure having a first circular cross-section; and
a second electrode in the form of a second mesh formed into a second tubular structure having a second circular cross-section, the second tubular structure being disposed inside the first tubular structure;
and wherein the first circular cross-section is smaller in diameter than the first reservoir opening such that the first and second electrodes fit through said first reservoir opening.

10. The apparatus of claim 8 wherein the reservoir and the base each have cross-sections that are non-circular, and an exterior peripheral wall of the reservoir transitions smoothly to an exterior peripheral wall of the base substantially without a discontinuity between the base and the reservoir.

11. The apparatus of claim 7 further comprising:
a removable cap that threadingly engages a threaded opening in the reservoir at an end of the reservoir opposite the electrodes, the removable cap including the valve, such that a user can substantially fill the reservoir with liquid and turn the reservoir assembly cap-side down without liquid substantially spilling therefrom, and place the reservoir assembly into the reservoir receiving portion of the mop.

12. The apparatus of claim 7 wherein:
the valve actuator comprises a fixed pin that presses against the valve when the reservoir is seated within the reservoir receiving section;
the valve is contained within a cap on the reservoir receiving section; and
the cap has an O-ring that, as the reservoir is being seated within the reservoir receiving portion, forms a watertight seal with the reservoir receiving section before the fix pin opens the valve.

13. The apparatus of claim 7 further comprising:
a hand-actuated spray pump assembly that threadingly engages the threaded opening in the reservoir when the removable cap is removed, the spray pump assembly including a spray handle and a tube in fluid communication therewith, the tube extending into the reservoir.

14. A versatile sanitizing and cleaning apparatus comprising: a removable electrolyzing vessel having a reservoir and electrodes for electrolyzing a liquid solution within the reservoir thereby producing an electrolyzed solution within the electrolyzing vessel, the electrolyzing vessel having a first opening;
a mop having:
a mop head;
a handle connected to the mop head;
an electrolyzing vessel receiving portion that removably mates with and holds the electrolyzing vessel, and allows said electrolyzed solution to flow from the first opening of the electrolyzing vessel thereby withdrawing the electrolyzed solution from the electrolyzing vessel; and
a first hand-powered sprayer that sprays the electrolyzed solution away from the mop, thereby allowing a user to spray electrolyzed solution from the mop onto a floor surface to be cleaned.

15. The apparatus of claim 14 wherein:
the first opening of the electrolyzing vessel defines a threaded opening; and
the apparatus further comprises:
a threaded cap that screws onto the threaded opening of the electrolyzing vessel;
a valve within the threaded cap that is biased to a normally closed position;
a protuberance within the electrolyzing vessel receiving portion that, when the electrolyzing vessel is held within the electrolyzing vessel receiving portion of the mop, opens the valve thereby allowing the electrolyzed solution to be withdrawn from the electrolyzing vessel and flow through the passageway to the first hand-powered sprayer.

16. The apparatus of claim 14 further comprising:
an adapter having:
a female thread that mates with the threaded opening of the electrolyzing vessel; and
a male thread;
and wherein the apparatus further comprises:
a second hand-powered sprayer having a pump and a female thread that mates with the male thread of the adapter; and
a tube that extends from the pump into the electrolyzed liquid within the electrolyzing vessel when the female thread of the adapter is screwed onto the male thread of the electrolyzing vessel and the female thread of the second hand-powered sprayer is screwed onto the male thread of the adapter.

17. The apparatus of claim 14 wherein:
the electrolyzing vessel first opening is disposed at a top end thereof;
the reservoir has a second opening at a bottom end thereof;
the electrolyzing vessel further comprises a base which threadingly and removably engages the second opening; and
the electrodes are affixed to the base and extend up through the second opening into the reservoir when the base is engaged with the reservoir.

18. The apparatus of claim 17 wherein the base further comprises a controller for controlling application of electrical power to the electrodes.

19. The apparatus of claim 14 wherein:
the electrolyzing vessel has a curved groove formed in an end thereof; and
the mop handle has a rotatable collar thereon, the rotatable collar including an engagement tab such that the rotatable collar can be rotated causing the engagement tab to slide into the curved groove in the end of the electrolyzing vessel thereby holding the electrolyzing vessel in the electrolyzing vessel receiving portion.

20. The apparatus of claim 17 wherein the reservoir and the base each have respective concave grooves therein for at least partially receiving the mop handle.

21. A sanitizing and cleaning apparatus comprising: a mop comprising:
a mop head;
a handle connected to the mop head;
an electrical input port for receiving electrical input power;
an electrolyzing vessel mounted to the handle, the electrolyzing vessel having:
a reservoir; and
electrodes powered by said electrical input power for electrolyzing a liquid solution within the reservoir thereby producing an electrolyzed solution within the electrolyzing vessel, the electrolyzing vessel having a first opening;
an electrolyzing vessel holding portion that holds the electrolyzing vessel, and allows said electrolyzed solution to flow from the first opening of the electrolyzing vessel thereby withdrawing the electrolyzed solution from the electrolyzing vessel; and
a sprayer that receives the electrolyzed solution after the electrolyzed solution has been withdrawn from the first opening and sprays the electrolyzed solution away from the mop, thereby allowing a user to spray electrolyzed solution from the mop onto a floor surface to be cleaned.

22. The apparatus of claim 21 further comprising a timer integrated into the mop for allowing electrical input power to flow to said electrodes for a predetermined amount of time.
